(19) United States
(12) Patent Application Publication
(10) Pub. No.: US 2025/0109447 A1
(43) Pub. Date: Apr. 3, 2025

(54) CRISPR-CAS-BASED COMPOSITION FOR SALMONELLA DETECTION AND SALMONELLA DETECTION METHOD USING SAME

(71) Applicant: PULMUONE CO., LTD., Eumseong-gun, Chungcheongbuk-do (KR)

(72) Inventors: Sang Yong LEE, Cheongju-si, Chungcheongbuk-do (KR); Young Min PARK, Cheongju-si, Chungcheongbuk-do (KR); Sang Gu KIM, Cheongju-si, Chungcheongbuk-do (KR); Sang Yoon LEE, Cheongju-si, Chungcheongbuk-do (KR); Hyo Yul LEE, Eumseong-gun, Chungcheongbuk-do (KR)

(21) Appl. No.: 18/841,241

(22) PCT Filed: Feb. 16, 2023
(86) PCT No.: PCT/KR2023/002300

§ 371 Date: Aug. 23, 2024

(30) Foreign Application Priority Data
Feb. 23, 2022 (KR) 10-2022-0023357

(51) Int. Cl. C12Q 1/689; C12N 9/22; C12N 15/11; C12Q 1/6844

(52) U.S. Cl. CPC C12Q 1/689; C12N 9/22; C12N 15/11; C12Q 1/6844; C12N 2310/20

(57) ABSTRACT

The present invention relates to a CRISPR-Cas based composition for detecting Salmonella and a method for detecting Salmonella using the same, more specifically, to a composition for detecting Salmonella comprising primers capable of specifically amplifying Salmonella through isothermal amplification, guide RNA, and CRISPR-Cas proteins, and a method for detecting Salmonella using the same.

### TECHNICAL FIELD

This application claims priority to Korean Patent Application No. 10-2022-0023357, filed on Feb. 23, 2022, and the entire disclosure of which is incorporated herein by reference.

The present invention relates to a composition for detecting Salmonella based on CRISPR-Cas and a method for detecting Salmonella using the same, more specifically to a composition for detecting Salmonella comprising a primer pair, guide RNA, and CRISPR-Cas protein capable of specifically amplifying Salmonella through isothermal amplification, and a method for detecting Salmonella using the same.

### BACKGROUND OF THE INVENTION

Salmonella is one of the most frequent causative agents of food poisoning both domestically and internationally. With the recent increase in group catering, the number of patients per incident has risen, making it a pathogen of significant public health concern. The genus Salmonella forms a single group based on DNA-DNA hybridization, showing 99% homology with Escherichia coli. According to the proposal of the World Health Organization (WHO) Collaborating Centre, it is classified into two species [Salmonella enterica and Salmonella bongori (V)] and six subspecies [Salmonella enterica subsp. enterica (I), Salmonella enterica subsp. salamae (II), Salmonella enterica subsp. arizonae (IIIa), Salmonella enterica subsp. diarizonae (IIb), Salmonella enterica subsp. houtenae (IV), Salmonella enterica subsp. indica (VI)].

The serotypes of Salmonella have been identified based on the Kaufmann-White classification, which relies on somatic antigens (O) and flagellar antigens (Phase I, II flagellin; H). To date, 46 O serotypes and 114 H serotypes have been identified, with a total of 2,523 different serotypes recognized. Identifying Salmonella serotypes is fundamental in epidemiological studies. Traditional serological methods require the availability of all sera for serotype identification and the acquisition of antisera for new serotypes as they emerge. Consequently, identification is performed in a limited number of accredited laboratories, and results may vary depending on the expressed phenotype. Additionally, unidentified isolates must be sent to the Pasteur Institute for serotype identification, which is cumbersome.

Meanwhile, Salmonella is a representative pathogenic microorganism causing food poisoning and is the causative agent of typhoid and paratyphoid fever, which are classified as first-class notifiable infectious diseases. Salmonella is a Gram-negative, non-spore-forming, facultatively anaerobic bacillus, classified into over 2,800 serotypes. Among these, Salmonella enterica is the representative species causing food poisoning, accounting for about 50% of disease occurrences. Notably, Salmonella enterica subsp. enterica I accounts for 99% of all human Salmonella infections.

According to the data on the incidence of food poisoning by causative agents from the Food Poisoning Statistics System of the Ministry of Food and Drug Safety of Korea (2002-2014), the number of food poisoning cases caused by Salmonella has been steadily increasing each year.

Therefore, there is a need to develop a method that can rapidly and accurately detect Salmonella strains, such as subtypes and serotypes, which frequently cause food poisoning.

### SUMMARY OF INVENTION

### Problems to be Solved

Accordingly, the present inventor has discovered the present invention by developing a CRISPR-Cas-based rapid detection method that can simultaneously target the top 10 serotypes (88.8% of all serotypes) among 32 serotypes of Salmonella isolated from domestic food, clinical, and environmental sources from 2012 to 2018, using a primer pair consisting of the nucleotide sequences of SEQ ID NO: 1 and SEQ ID NO: 2, and optionally guide RNA and CRISPR-Cas proteins, to detect Salmonella with very high sensitivity and specificity.

Therefore, an object of the present invention is to provide a primer pair for detecting Salmonella, comprising a forward primer consisting of the nucleotide sequence of SEQ ID NO: 1 and a reverse primer consisting of the nucleotide sequence of SEQ ID NO: 2.

Another object of the present invention is to provide a composition for detecting Salmonella, comprising the primer pair of SEQ ID NOs: 1 and 2, guide RNA, and an endonuclease.

Another object of the present invention is to provide a composition for detecting Salmonella, consisting of the primer pair of SEQ ID NOs: 1 and 2, guide RNA, and an endonuclease.

Another object of the present invention is to provide a composition for detecting Salmonella, consisting essentially of the primer pair of SEQ ID NOs: 1 and 2, guide RNA, and an endonuclease.

Another object of the present invention is to provide a kit for detecting Salmonella, comprising the composition.

Another object of the present invention is to provide a method for detecting Salmonella, comprising the following steps:
(a) isolating DNA from a sample;
(b) amplifying the DNA using the primer pair; and
(c) detecting the amplified product.

Another object of the present invention is to provide a composition for diagnosing food poisoning.

Another object of the present invention is to provide a use of the primer pair, guide RNA, and endonuclease for manufacturing a composition for detecting Salmonella.

Another object of the present invention is to provide a use of a composition for manufacturing a composition for diagnosing food poisoning.

Another object of the present invention is to provide a method for diagnosing food poisoning, comprising the following steps:
(a) isolating DNA from a sample;
(b) amplifying the DNA using the primer pair;
(c) detecting the amplified product; and
(d) diagnosing food poisoning if the amplified product is detected in step (c).

However, the problems of the present invention as described above are exemplary and do not limit the scope of the present invention. Further, other objects and advantages of the present invention will become more apparent from the detailed description, claims, and drawings of the invention.

### Means for Solving the Problem

To achieve the aforementioned objectives of the present invention, the invention provides a primer pair for detecting Salmonella, comprising a forward primer consisting of the nucleotide sequence of SEQ ID NO: 1 and a reverse primer consisting of the nucleotide sequence of SEQ ID NO: 2.

To achieve another objective of the present invention, the invention provides a composition for detecting Salmonella, comprising the primer pair of SEQ ID NOs: 1 and 2, guide RNA (gRNA), and an endonuclease.

To achieve another objective of the present invention, the invention provides a composition for detecting Salmonella, consisting of the primer pair of SEQ ID NOs: 1 and 2, guide RNA (gRNA), and an endonuclease.

To achieve another objective of the present invention, the invention provides a composition for detecting Salmonella, consisting essentially of the primer pair of SEQ ID NOs: 1 and 2, guide RNA (gRNA), and an endonuclease.

To achieve another objective of the present invention, the invention provides a kit for detecting Salmonella, comprising the composition.

To achieve another objective of the present invention, the invention provides a method for detecting Salmonella, comprising the following steps:
(a) isolating DNA from a sample;
(b) amplifying the DNA using the primer pair; and
(c) detecting the amplified product.

To achieve another objective of the present invention, the invention provides a composition for diagnosing food poisoning.

To achieve another objective of the present invention, the invention provides the use of a primer pair, guide RNA (gRNA), and an endonuclease for manufacturing a composition for detecting Salmonella.

To achieve another objective of the present invention, the invention provides the use of a composition for manufacturing a composition for diagnosing food poisoning.

To achieve another objective of the present invention, the invention provides a method for diagnosing food poisoning, comprising the following steps:
(a) isolating DNA from a sample;
(b) amplifying the DNA using the primer pair;
(c) detecting the amplified product; and
(d) diagnosing food poisoning if the amplified product is detected in step (c).

Unless otherwise defined, all technical and scientific terms used herein have the same meaning as commonly understood by one of ordinary skill in the art. The following references provide one example of the general definitions of many terms used in this specification: Singleton et al., DICTIONARY OF MICROBIOLOGY AND MOLECULAR BIOLOGY (2nd ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY.

Hereinafter, the present invention will be described in detail.

The present invention provides a primer pair for detecting Salmonella, comprising a forward primer consisting of the nucleotide sequence of SEQ ID NO: 1 and a reverse primer consisting of the nucleotide sequence of SEQ ID NO: 2.

In the present invention, the term “primer” refers to a short nucleic acid sequence with a free 3′ hydroxyl group that can form a base pair with a complementary template of E. coli nucleic acid and functions as a starting point for template strand replication.

The primer pair specifically amplifies the treC gene of Salmonella, resulting in a 202 bp amplified product, and Salmonella can be detected through the analysis of the amplified product.

In the present invention, the Salmonella may be characterized as Salmonella Enterica or its serovar.

In the present invention, the serovar may be selected from the group consisting of Salmonella enteritidis, Salmonella typhimurium, Salmonella braenderup, Salmonella typhi, Salmonella thompson, Salmonella taintpaul, Salmonella paratyphi, Salmonella virchow, Salmonella senftenberg, Salmonella newport, Salmonella montevideo, Salmonella london, and Salmonella Infantis.

According to one embodiment of the present invention, the primer pair according to the present invention was confirmed to amplify the genes of 13 serovars and 16 standard strains of Salmonella. Additionally, the primer pair does not amplify the genes of other microorganisms causing food poisoning, indicating very high detection specificity.

The amplification can be performed by any method used in the art to amplify the target (gene, etc.), for example, by Recombinase Polymerase Amplification (RPA), but is not limited thereto.

The amplified product may be, for example, an amplicon, but is not limited thereto.

In this specification, the term “Recombinase Polymerase Amplification (RPA)” refers to a technique that can confirm DNA and RNA amplification, which, unlike conventional PCR, uses DNA-binding proteins and recombinase to quickly and accurately amplify target sequences. RPA is similar to conventional PCR but can amplify specific genes under isothermal conditions without temperature changes, reducing reaction time, and does not require special equipment, making it cost-effective and portable for field testing. Recombinase Polymerase Amplification (RPA) uses bacteriophage T4 recombinase to cause the denaturation of double-stranded DNA and simultaneously amplifies specific DNA using DNA polymerase and specific primers. This method can amplify DNA of specific sequences using a target template and a pair of primers (oligonucleotides) like PCR, but unlike PCR, it can induce an amplification reaction under isothermal conditions within a temperature range of 37° C.-42° C. Currently, RPA has been developed to produce amplified products very quickly and is widely recognized for its application in detecting specific pathogens through specific gene amplification.

The length of RPA amplified products is recommended not to exceed 500 nucleotides (nt) and generally has a length between 100 and 250 nt. The primer length for RPA is recommended to be at least 30 nucleotides, unlike PCR, and generally uses a length of 32 to 35 nt. Additionally, RPA primers are not sensitive to the GC content (GC %) and Tm (melting temperature) value, so RPA primers may have the value of 20-70% of GC % and 50-100° C. of Tm (Analyst, 2019, 144, 31).

The present invention also provides a composition for detecting Salmonella, comprising the primer pair of SEQ ID NOs: 1 and 2, guide RNA (gRNA), and an endonuclease.

In the present invention, the guide RNA may be crRNA or gRNA. crRNA is called CRISPR RNA. Additionally, gRNA refers to guide RNA. crRNA and gRNA may be single-stranded RNA. Additionally, crRNA can bind to tracrRNA to activate CRISPR-associated proteins, and crRNA can be used in a form bound to tracrRNA. In this case, crRNA may have a sequence complementary to a gene sequence specifically present in Salmonella. Additionally, gRNA can also bind to a gene sequence specifically present in Salmonella to activate CRISPR-associated proteins. The crRNA or gRNA may be RNA consisting of 15 to 40 nucleotides. In one embodiment, crRNA or gRNA may consist of 24 nucleotides. Additionally, crRNA or gRNA may include additional sequences at the 3′ end to activate CRISPR-associated proteins such as Cas9, Cas12, or Cas13. In one embodiment, the gRNA may be consisting of the nucleotide sequence of SEQ ID NO: 3.

In the present invention, the endonuclease may be a CRISPR-associated protein. The term ‘CRISPR-associated protein’ used in the present invention refers to an enzyme that can recognize and cleave nucleic acids such as DNA or RNA when they have double-stranded or single-stranded forms (dsDNA/RNA and ssDNA/RNA). Specifically, they can recognize and cleave double-stranded or single-stranded nucleic acids bound to crRNA or gRNA.

In one specific example of the present invention, the endonuclease may be activated by recognizing that gRNA is bound to the target site. Additionally, as the endonuclease function is activated, it may have exonuclease activity that can non-specifically cleave double-stranded and/or single-stranded DNA and/or RNA. Additionally, CRISPR-associated proteins such as Cas12a can exhibit non-specific exonuclease activity once activated. In such cases, DNA and RNA can be non-specifically cleaved.

Therefore, in one specific example of the composition of the present invention, Salmonella can be detected by the fluorescence emission of a reporter bound to a fluorescent substance, which is cleaved by a non-specific nuclease activated by the binding of crRNA or gRNA to a specific target site, more specifically the treC gene of Salmonella.

Specifically, one specific example of the CRISPR-associated protein may be selected from the group consisting of Cas1, Cas1B, Cas2, Cas3, Cas4, Cas5, Cas6, Cas7, Cas8, Cas9, Cas10, Cas12a, Cas12b, Cas12c, Cas12d, Cas12e, Cas12g, Cas12h, Cas12i, Cas13a, Cas13b, Cas13c, Cas13d, Cas14, Csy1, Csy2, Csy3, Cse1, Cse2, Csc1, Csc2, Csa5, Csn2, CsMT2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4, preferably selected from the group consisting of Cas9, Cas10, Cas12a, Cas12b, Cas12c, Cas12d, Cas12e, Cas12g, Cas12h, Cas12i, Cas13a, Cas13b, Cas13c, and Cas13d, more preferably selected from the group consisting of Cas12a, Cas12b, Cas12c, Cas12d, Cas12e, Cas12g, Cas12h, and Cas12i, and most preferably Cas12a (Cpf1).

In one aspect of the present invention, the composition may further comprise a reporter gene.

In the present invention, the reporter gene is preferably bound to a fluorescent substance, and the fluorescent substance may be selected from the group consisting of FAM, TET, JOE, YAKYE, HEX, CY3, ATTO550, TAM, ROX, TxRed, CY35, LC610, LC640, ATTO647N, CY5, VIC, evergreen dye, and ATTO680, but is not limited thereto.

In one aspect of the present invention, the composition may be used in a method for detecting Salmonella by an isothermal amplification reaction and a subsequent CRISPR gene editing reaction.

Specifically, if the target site is present in the amplified product amplified through the primer pair consisting of the nucleotide sequences of SEQ ID NOs: 1 and 2, the guide RNA binds complementarily to the target site, and the endonuclease, preferably the CRISPR-associated protein, is activated to cleave the sequence of the reporter gene. As a result, the fluorescent substance bound to the reporter gene emits fluorescence, and Salmonella can be detected through this.

The present invention also provides a kit for detecting Salmonella, comprising the composition.

The kit may be a kit comprising essential elements necessary for performing Salmonella detection, and in addition to the composition, it may include test tubes or other suitable containers, reaction buffers (various pH and magnesium concentrations), deoxynucleotides (dNTPs), enzymes such as Taq-polymerase, DNase, RNAse inhibitors, DEPC-water, and sterile water.

The present invention also provides a method for detecting Salmonella, comprising the following steps:
(a) isolating DNA from a sample;
(b) amplifying the DNA using the primer pair; and
(c) detecting the amplified product.

In the present invention, the “sample” refers to a material or organism used for testing, examination, or analysis, and is not limited in type, but may be a food or biological sample suspected of containing Salmonella.

In the present invention, the biological sample may include tissue, cells, whole blood, serum, plasma, cerebrospinal fluid, colostrum, joint fluid, saliva, feces, cell culture supernatant, etc., but is not limited thereto.

The isolation of DNA from the sample can be performed using conventional known DNA isolation methods. If the starting material is gDNA, the isolation of gDNA can be performed according to conventional methods known in the art, and if the starting material is mRNA, it can be synthesized into cDNA using reverse transcriptase. For example, the DNA isolation is not particularly limited as long as it is a method for isolating genomic DNA (gDNA) from a sample. For example, commercially available DNA isolation kits such as the DNeasy mini kit from Qiagen can be used.

In the present invention, the “amplification” may be selected from the group consisting of PCR, RT-PCR, and isothermal amplification, preferably isothermal amplification.

The “isothermal amplification” refers to a method for amplifying a target nucleic acid sequence (template) under constant reaction temperature conditions, and may be selected from the group consisting of recombinase polymerase amplification (RPA), loop-mediated isothermal amplification (LAMP), helicase-dependent amplification (HDA), rolling circle amplification (RCA), multiple displacement amplification (MDA), strand displacement amplification (SDA), and nucleic acid sequence-based amplification (NASBA), preferably recombinase polymerase amplification (RPA).

The detection of the amplified product can be performed through capillary electrophoresis, DNA chips, gel electrophoresis, radiometric measurement, fluorescence measurement, or phosphorescence measurement, but is not limited thereto.

As one method for detecting the amplified product, for example, capillary electrophoresis can be performed. Capillary electrophoresis can be performed using, for example, an ABi Sequencer. Additionally, gel electrophoresis can be performed, and gel electrophoresis can use agarose gel electrophoresis or acrylamide gel electrophoresis depending on the size of the amplified product. Additionally, the fluorescence measurement method can label the 5′-end of the primer with Cy-5 or Cy-3 to perform PCR, and the target sequence can be detected with a fluorescent label, and the labeled fluorescence can be measured using a fluorescence detector. Additionally, the radiometric measurement method can label the amplified product by adding radioactive isotopes such as ³²P or ³⁵S to the PCR reaction solution during PCR, and measure the radioactivity using a radiometric measuring device, such as a Geiger counter or a liquid scintillation counter. Additionally, the RPA amplified product can be obtained and separated by methods such as agarose gel electrophoresis, and the presence of DNA corresponding to the length of the DNA polymerized by the primer set used, specifically DNA with a length of 202 bp, can be confirmed to detect Salmonella.

In one aspect of the present invention, after step (b), the method may further comprise the step of mixing the amplified product with a composition comprising guide RNA, endonuclease, and a reporter gene.

In this case, the presence of Salmonella can be confirmed by detecting the fluorescence reaction of the reporter gene according to the gene editing reaction by the guide RNA and endonuclease.

The present invention also provides a composition for diagnosing food poisoning.

The present invention provides the use of a primer pair, guide RNA (gRNA), and an endonuclease for manufacturing a composition for detecting Salmonella.

The present invention provides the use of a composition for manufacturing a composition for diagnosing food poisoning.

The present invention provides a method for diagnosing food poisoning, comprising the following steps:
(a) isolating DNA from a sample;
(b) amplifying the DNA using the primer pair;
(c) detecting the amplified product; and
(d) diagnosing food poisoning if the amplified product is detected in step (c).

In one embodiment, the present invention provides a method for diagnosing and treating food poisoning in a sample, comprising the following steps:
(i) isolating DNA from a sample;
(ii) amplifying the DNA using the primer pair;
(iii) detecting the amplified product;
(iv) diagnosing food poisoning if the amplified product is detected in step (iii); and
(v) administering a therapeutic drug or performing surgery to treat the disease in the diagnosed sample.

The methods comprising steps (i) to (v) are understood in accordance with the methods comprising steps (a) to (d) described above.

Step (v) is a step of treating the disease by administering a therapeutic drug such as ciprofloxacin, azithromycin, or ceftriaxone, or by surgical means to the sample diagnosed with the disease in step (iv).

The “treatment” in the present invention comprehensively refers to improving food poisoning or its symptoms, and may include curing, substantially preventing, or improving the condition, and may include alleviating, curing, or preventing one or more symptoms derived from the disease, but is not limited thereto.

The “sample” in the present invention is obtained from a sample suspected of having the disease, and may be selected from the group consisting of cells, tissues, blood, serum, plasma, saliva, sputum, mucous fluid, and urine, but is not limited thereto. The “sample” or “subject” may be an animal, preferably a mammal, particularly an animal including a human, and may be cells, tissues, organs, etc., derived from the animal. The sample may be a patient in need of the therapeutic effect.

The term “diagnosis” as used herein includes determining whether a subject currently has a specific disease or condition, or determining the prognosis of a subject with a specific disease or condition.

The term “comprising” as used herein is synonymous with “including” or “characterized by,” and does not exclude additional components or steps not specifically mentioned in the composition or method according to the present invention. The term “consisting of” means excluding additional elements, steps, or components not specifically described. The term “consisting essentially of” means that the composition or method may include additional elements or steps that do not materially affect the basic characteristics of the composition or method.

The composition comprising the primer pair and guide RNA provided by the present invention can specifically detect Salmonella, and thus, using this, Salmonella in a biological sample to be diagnosed (e.g., feces, blood, etc.) can be quickly and accurately detected, allowing for the rapid and accurate diagnosis of diseases caused by Salmonella infection, specifically food poisoning.

### Effects of the Invention

The primer pair provided by the present invention, or the use of the primer pair and guide RNA, enables the rapid detection of Salmonella bacteria in a sample with very high sensitivity and specificity. Additionally, it allows for the simultaneous detection of 13 serotypes of Salmonella bacteria that are clinically significant in causing diseases, making it highly applicable in both food and clinical fields.

### BRIEF DESCRIPTION OF THE DRAWINGS

FIG. 1a to if are diagrams sequentially illustrating the gRNA selection process for detecting Salmonella bacteria.

FIGS. 2a and 2b show the results of RPA primers for the selected three gRNA candidates and evaluating the amplification capability of genes isolated from 13 serotypes and 16 standard Salmonella strains.

FIGS. 3a and 3b illustrate the results of amplifying genes isolated from 13 serotypes and 16 standard Salmonella strains using the selected gRNA target RPA primer pairs, and detecting the amplified products using gRNA and CRISPR-Cas12a.

FIG. 4 shows the results of evaluating detection specificity by performing detection reactions on various foodborne pathogens indicated in the drawings using the selected gRNA target RPA primer pairs, gRNA, and CRISPR-Cas12a.

FIG. 5 shows the results of evaluating the detection sensitivity for Salmonella bacteria by applying PCR with the selected gRNA RPA primer pairs to detect Salmonella enterica subsp. enterica serovar Enteritidis (ATCC 13076) in order to verify the detection sensitivity of the selected gRNA and RPA primers.

### DETAILED DESCRIPTION OF THE INVENTION

Hereinafter, preferred embodiments are provided to aid in the understanding of the present invention. However, the following embodiments are provided merely to facilitate understanding of the present invention and are not intended to limit the scope of the invention.

### Example 1: Primary Selection of gRNA for Salmonella Detection

The core of research in the diagnostic field using CRISPR gene-editing technology comprises the applied Cas protein and gRNA. In this study, we selected cpf1 (Cas12a), which has activity on dsDNA and is known to require a T-rich PAM (TTTV) within the target.

Through bioinformatics techniques, we secured 75,651 gRNA candidates with T-rich PAM+24 nt for the Salmonella enterica subsp. enterica serovar Typhimurium str. LT2 ref. genome (NC_003197).

Among 32 serotypes of Salmonella isolated from domestic food, clinical, and environmental sources from 2012 to 2018, we identified 9 gRNA candidates that commonly match 9 serotypes (S. thompson, S. montevideo, S. braenderup, S. enteretidis, S. infantis, S. Livingstone, S. Heidelberg, S. Virchow, S. Typhimurium) through gRNA matching that can simultaneously identify the top 10 serotypes (S. Thompson, S. Montevideo, S. Braenderup, S. Enteretidis, S. Infantis, S. Livingstone, S. Heidelberg, S. Virchow, S. Reading, S. Typhimurium), which account for 88.8% of all strains (Reference: Food Poisoning Bacteria Characteristic Analysis Report—Salmonella spp. —2019, Ministry of Food and Drug Safety, Food and Drug Safety Evaluation Institute).

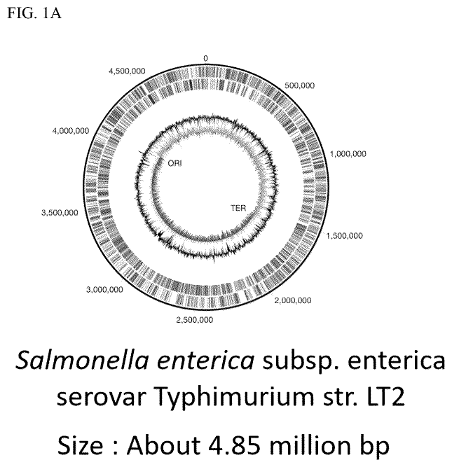

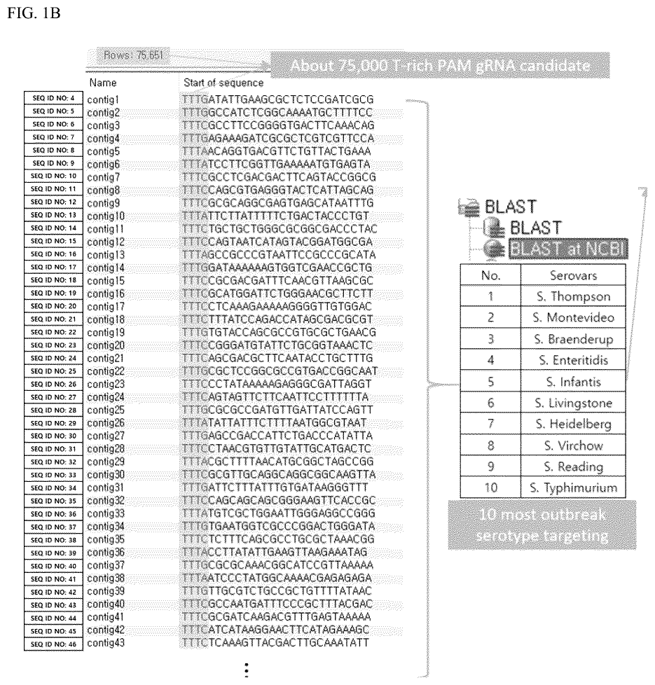

We then filtered these to 3 gRNA candidates with good secondary structures that map to genes on the Salmonella enterica subsp. enterica serovar Typhimurium str. LT2 ref. genome (FIGS. 1a to f).

### Example 2: Design of RPA Primers and Secondary Selection of gRNA

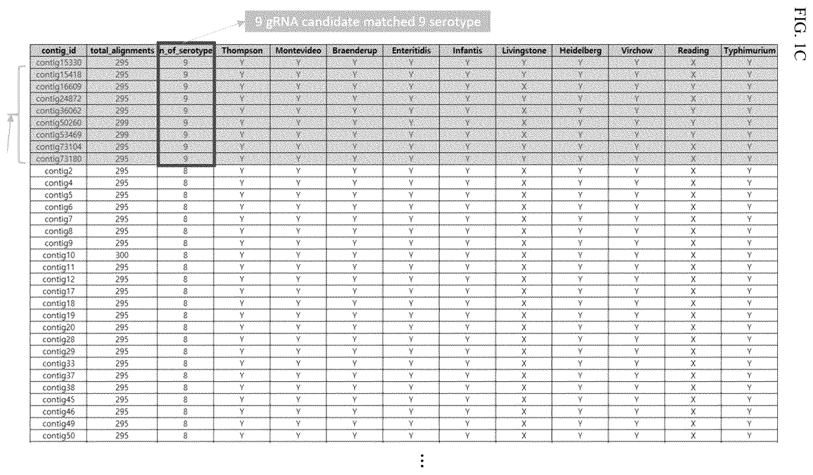

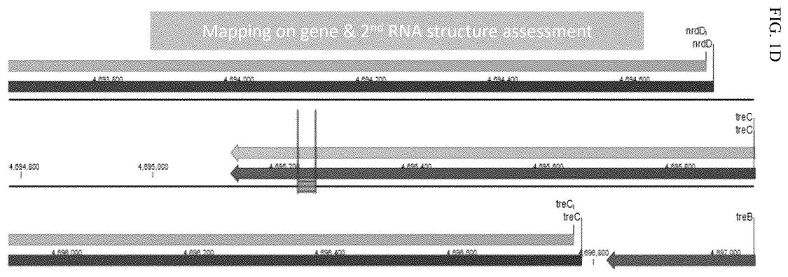

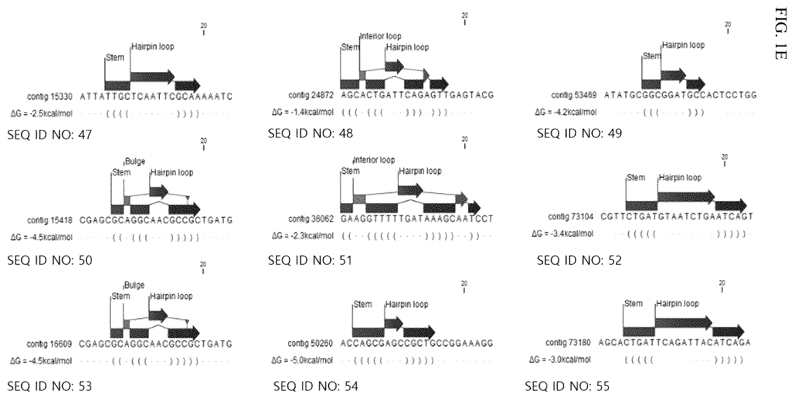

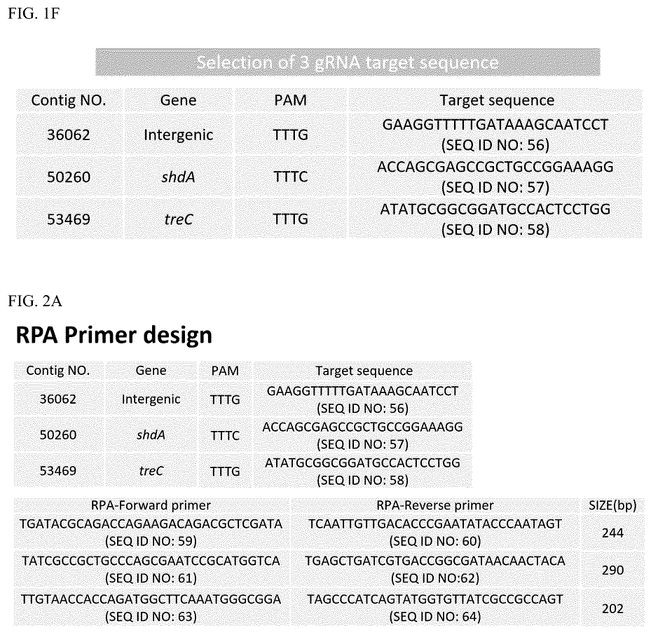

For the 3 gRNA candidates selected in Example 1, RPA primers were designed, and RPA amplification experiments were conducted using 13 serotypes and 16 standard Salmonella strains. 1 gRNA (treC) that showed amplification across all samples was selected (FIGS. 2a and b).

### Example 3: Detection Experiment of Salmonella Using RPA Primers, gRNA, and CRISPR-Cas12a

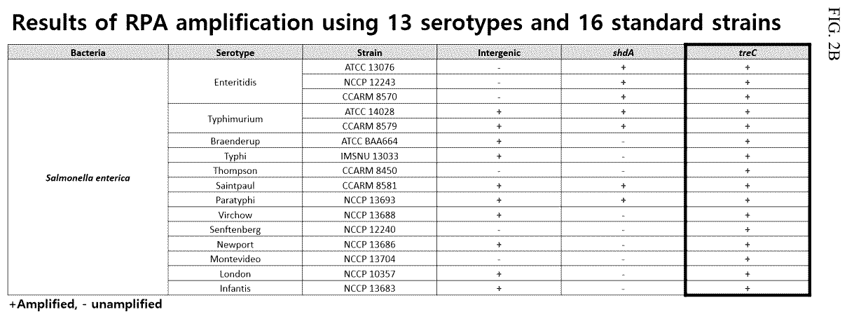

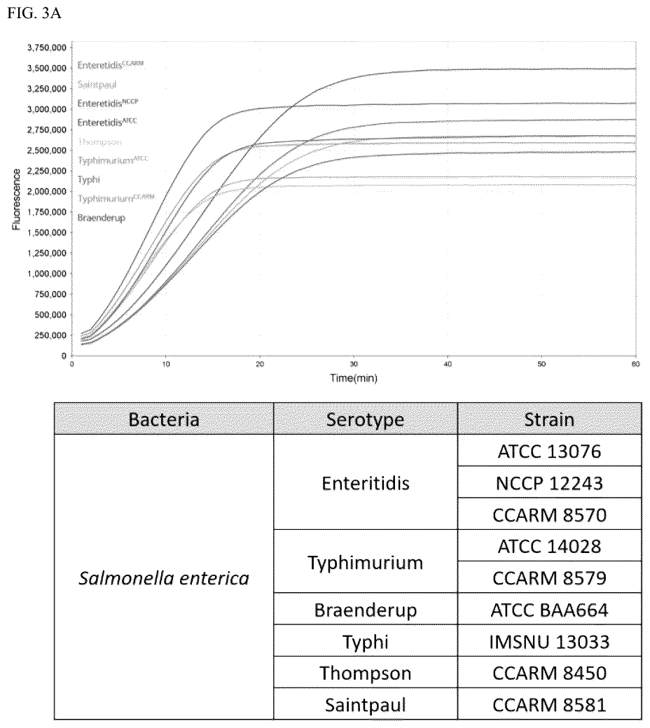

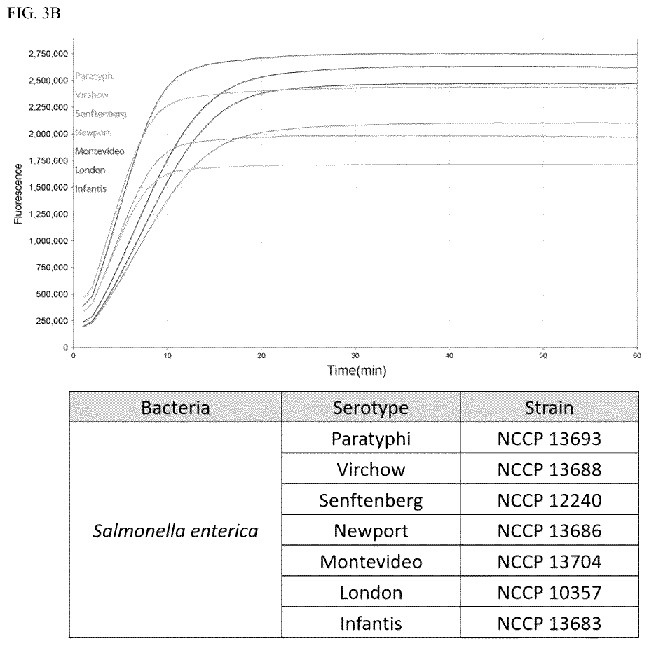

Using the RPA primer pairs targeting the selected gRNA, we conducted reactions applying gRNA and CRISPR-Cas12a on the amplicons from 13 serotypes and 16 standard Salmonella strains. The detailed reaction conditions are shown in Table 1. The mixture prepared according to the protocol was measured every minute using a 37° C. isothermal fluorescence detector, and the results are shown in FIGS. 3a and b.

TABLE 1

| Components | Stock conc. | | Final conc.(Tube) | |
|---|---|---|---|---|
| buffer | 10 x | | 1 x | |
| gRNA | 10 | μM | 0.5 | μM |
| Cpf1 | 1 | μM | 0.05 | Mm |
| ssDNA reporter | 10 | μM | 0.5 | μM |
| Rnase inhibitor | 40 | U/μL | 10 | U |

| | |
|---|---|
| Sterile water | Add to total reaction volume of 18 μL |
| RPA amplicon | Add 2 μL |

As shown in FIG. 3, the primer pairs and gRNA selected in Example 2 demonstrated rapid detection speed and good fluorescence values for 13 serotypes and 16 standard Salmonella strains.

### Example 4: Evaluation of Detection Specificity Using RPA Primers, gRNA, and CRISPR-Cas12a

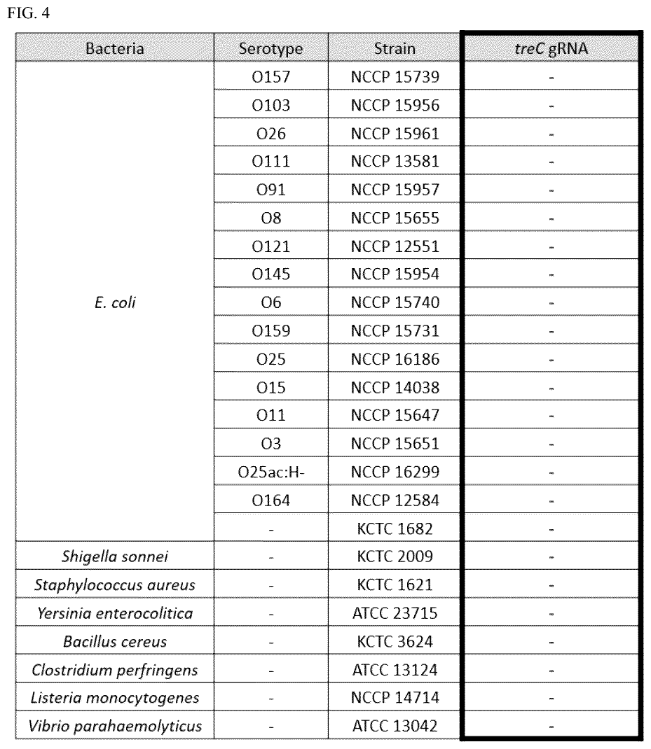

To verify that the gRNA selected in Example 2 can specifically detect only Salmonella, DNA extraction was performed on 24 representative non-Salmonella foodborne pathogens. Amplification was conducted using the RPA primer pairs selected in Example 2, and the reaction mixture was prepared according to the protocol in Table 1 using the gRNA selected in Example 2. Measurements were taken every minute for 1 hour using a 37° C. isothermal fluorescence detector, and the results are shown in FIG. 4.

As shown in FIG. 4, the specificity test results for 24 representative non-Salmonella foodborne pathogens confirmed that no detection occurred, indicating that the selected gRNA and RPA primers specifically detect only Salmonella.

Additionally, diagnosis was possible at 37° C. from RPA (approximately 20 minutes) to CRISPR detection (approximately 20 minutes), confirming that the RPA primer pairs (Sequence Nos. 1 and 2) and gRNA (Sequence No. 3) composition can be utilized for rapid diagnosis of Salmonella.

### Example 5: Evaluation of Detection Sensitivity Using RPA Primers, gRNA, and CRISPR-Cas12a

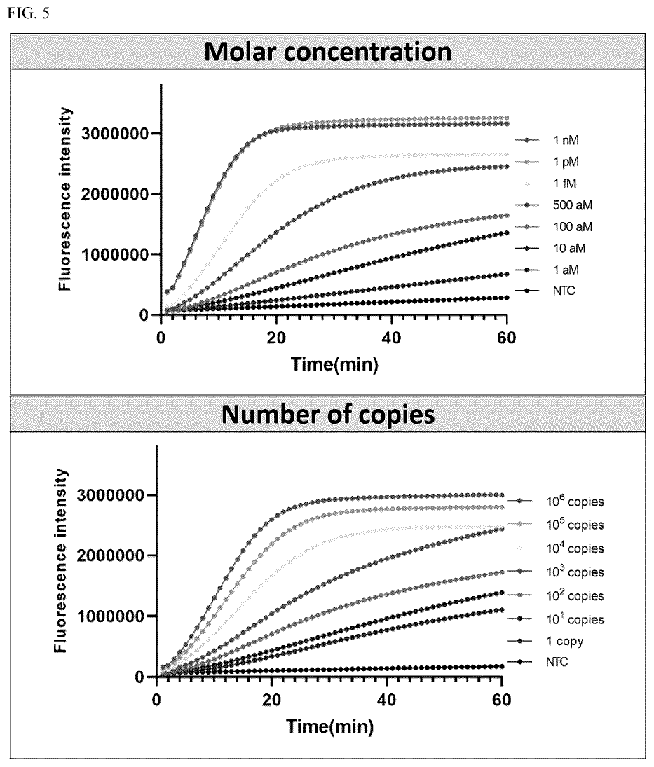

To verify the detection sensitivity of the selected gRNA and RPA primers for Salmonella, amplicons for Salmonella enterica subsp. enterica serovar Enteritidis (ATCC 13076) were obtained through PCR using the RPA primer pairs selected in Example 2. Standard plasmid DNA was then prepared from these amplicons. The prepared plasmid DNA was serially diluted to concentrations of 1 nM, 1 pM, 1 fM, 500 aM, 100 aM, 10 aM, 1 aM, and 10⁶ copies, 105 copies, 104 copies, 103 copies, 10² copies, 10 copies, 1 copy. RPA and CRISPR detection sensitivity evaluations were conducted using the RPA primer pairs and gRNA selected in Example 2, and the results are shown in FIG. 5.

As shown in FIG. 5, it was confirmed that Salmonella can be detected with high sensitivity at levels of 1 aM (10⁻¹⁸ molar) and 1 copy within approximately 20 minutes after the initiation of the CRISPR-Cas reaction.

### INDUSTRIAL APPLICABILITY

The primer pair or the primer pair and guide RNA, provided by the present invention, have the ability to rapidly detect Salmonella in a sample with very high sensitivity and specificity. Additionally, it can simultaneously detect 13 serotypes of Salmonella that frequently cause clinical diseases, making it highly applicable in the food and clinical fields, thus having high industrial applicability.

1.-23. (canceled)

24. A composition for detecting Salmonella spp., comprising a primer pair capable of amplifying the sequence ATATGCGGCGGATGCCACTCCTGG or a sequence complementary thereto in the treC gene of Salmonella spp., guide RNA comprising the nucleotide sequence of SEQ ID NO: 3, and endonuclease, wherein the endonuclease is selected from the group consisting of Cas12, Cas13 and Cas14.

25. The composition of claim 24, wherein the primer pair comprises a forward primer consisting of the nucleotide sequence of SEQ ID NO: 1 and a reverse primer consisting of the nucleotide sequence of SEQ ID NO: 2.

26. The composition of claim 24, wherein the Salmonella spp. is Salmonella enterica or a serovar thereof.

27. The composition of claim 26, wherein the serovar is selected from the group consisting of Salmonella enteritidis, Salmonella typhimurium, Salmonella braenderup, Salmonella typhi, Salmonella thompson, Salmonella saintpaul, Salmonella Paratyphi, Salmonella virchow, Salmonella senftenberg, Salmonella newport, Salmonella montevideo, Salmonella london, and Salmonella infantis.

28. The composition of claim 24, wherein the endonuclease is selected from the group consisting of Cas12a, Cas12b, Cas12c, Cas12d, Cas12e, Cas12g, Cas12h, Cas12i, Cas13a, Cas13b, Cas13c, Cas13d, and Cas14.

29. The composition of claim 24, further comprising a reporter gene.

30. The composition of claim 29, wherein the reporter gene is conjugated with a fluorescent substance.

31. The composition of claim 30, wherein the fluorescent substance is selected from the group consisting of FAM, TET, JOE, YAKYE, HEX, CY3, ATTO550, TAM, ROX, TxRed, CY35, LC610, LC640, ATTO647N, CY5, VIC, evergreen dye, and ATTO680.

32. The composition of claim 24, wherein the composition detects Salmonella spp. through an isothermal amplification reaction followed by a CRISPR gene-editing reaction.

33. A kit for detecting Salmonella spp., comprising the composition of claim 24.

34. A method for detecting Salmonella spp., comprising the steps of:
(a) isolating a nucleic acid from a sample;
(b) amplifying the nucleic acid using a primer pair capable of amplifying the sequence ATATGCGGCGGATGCCACTCCTGG or a sequence complementary thereto in the treC gene of Salmonella spp., and mixing the amplified product with a composition comprising a guide RNA, an endonuclease, and a reporter gene, wherein the endonuclease is selected from the group consisting of Cas12, Cas 13 and Cas 14; and
(c) detecting the amplified product.

35. The method of claim 34, wherein the amplification is an isothermal amplification.

36. The method of claim 35, wherein the isothermal amplification is recombinase polymerase amplification (RPA).

37. The method of claim 34, wherein the sample is a food or biological sample.

38. A composition for diagnosing food poisoning, comprising the composition of claim 24.

39. The composition of claim 38, wherein the food poisoning is caused by Salmonella spp. infection.


SEQUENCE LISTING (64 sequences)

SEQ ID NO: 1 - DNA, 32 bp, synthetic construct
  misc_feature 1..32: note = treC_forward primer
  source 1..32: mol_type = other DNA
ttgtaaccaccagatggcttcaaatgggcgga

SEQ ID NO: 2 - DNA, 32 bp, synthetic construct
  misc_feature 1..32: note = treC_reverse primer
  source 1..32: mol_type = other DNA
tagcccatcagtatggtgttatcgccgccagt

SEQ ID NO: 3 - RNA, 24 nt, synthetic construct
  misc_feature 1..24: note = treC_gRNA
  source 1..24: mol_type = other RNA
atatgcggcggatgccactcctgg

SEQ ID NO: 4 - DNA, 28 bp, Salmonella sp.
  source 1..28: mol_type = genomic DNA
tttgatattgaagcgctctccgatcgcg

SEQ ID NO: 5 - DNA, 28 bp, Salmonella sp.
  source 1..28: mol_type = genomic DNA
tttggccatctcggcaaaatgcttttcc

SEQ ID NO: 6 - DNA, 28 bp, Salmonella sp.
  source 1..28: mol_type = genomic DNA
tttcgccttccggggtgacttcaaacag

SEQ ID NO: 7 - DNA, 28 bp, Salmonella sp.
  source 1..28: mol_type = genomic DNA
tttgagaaagatcgcgctcgtcgttcca

SEQ ID NO: 8 - DNA, 28 bp, Salmonella sp.
  source 1..28: mol_type = genomic DNA
tttaacaggtgacgttctgttactgaaa

SEQ ID NO: 9 - DNA, 28 bp, Salmonella sp.
  source 1..28: mol_type = genomic DNA
tttatccttcggttgaaaaatgtgagta

SEQ ID NO: 10 - DNA, 28 bp, Salmonella sp.
  source 1..28: mol_type = genomic DNA
tttcgcctcgacgacttcagtaccggcg

SEQ ID NO: 11 - DNA, 28 bp, Salmonella sp.
  source 1..28: mol_type = genomic DNA
tttccagcgtgagggtactcattagcag

SEQ ID NO: 12 - DNA, 28 bp, Salmonella sp.
  source 1..28: mol_type = genomic DNA
tttcgcgcaggcgagtgagcataatttg

SEQ ID NO: 13 - DNA, 28 bp, Salmonella sp.
  source 1..28: mol_type = genomic DNA
tttattcttatttttctgactaccctgt

SEQ ID NO: 14 - DNA, 28 bp, Salmonella sp.
  source 1..28: mol_type = genomic DNA
tttctgctgctgggcgcggcgaccctac

SEQ ID NO: 15 - DNA, 28 bp, Salmonella sp.
  source 1..28: mol_type = genomic DNA
tttccagtaatcatagtacggatggcga

SEQ ID NO: 16 - DNA, 28 bp, Salmonella sp.
  source 1..28: mol_type = genomic DNA
tttagccgcccgtaattccgcccgcata

SEQ ID NO: 17 - DNA, 28 bp, Salmonella sp.
  source 1..28: mol_type = genomic DNA
tttggataaaaaagtggtcgaaccgctg

SEQ ID NO: 18 - DNA, 28 bp, Salmonella sp.
  source 1..28: mol_type = genomic DNA
tttccgcgacgatttcaacgttaagcgc

SEQ ID NO: 19 - DNA, 28 bp, Salmonella sp.
  source 1..28: mol_type = genomic DNA
tttcgcatggattctgggaacgcttctt

SEQ ID NO: 20 - DNA, 28 bp, Salmonella sp.
  source 1..28: mol_type = genomic DNA
tttcctcaaagaaaaaggggttgtggac

SEQ ID NO: 21 - DNA, 28 bp, Salmonella sp.
  source 1..28: mol_type = genomic DNA
tttctttatccagaccatagcgacgcgt

SEQ ID NO: 22 - DNA, 28 bp, Salmonella sp.
  source 1..28: mol_type = genomic DNA
tttgtgtaccagcgccgtgcgctgaacg

SEQ ID NO: 23 - DNA, 28 bp, Salmonella sp.
  source 1..28: mol_type = genomic DNA
tttccgggatgtattctgcggtaaactc

SEQ ID NO: 24 - DNA, 28 bp, Salmonella sp.
  source 1..28: mol_type = genomic DNA
tttcagcgacgcttcaatacctgctttg

SEQ ID NO: 25 - DNA, 28 bp, Salmonella sp.
  source 1..28: mol_type = genomic DNA
tttgcgctccggcgccgtgaccggcaat

SEQ ID NO: 26 - DNA, 28 bp, Salmonella sp.
  source 1..28: mol_type = genomic DNA
tttccctataaaaagagggcgattaggt

SEQ ID NO: 27 - DNA, 28 bp, Salmonella sp.
  source 1..28: mol_type = genomic DNA
tttcagtagttcttcaattcctttttta

SEQ ID NO: 28 - DNA, 28 bp, Salmonella sp.
  source 1..28: mol_type = genomic DNA
tttgcgcgccgatgttgattatccagtt

SEQ ID NO: 29 - DNA, 28 bp, Salmonella sp.
  source 1..28: mol_type = genomic DNA
tttatattatttcttttaatggcgtaat

SEQ ID NO: 30 - DNA, 28 bp, Salmonella sp.
  source 1..28: mol_type = genomic DNA
tttgagccgaccattctgacccatatta

SEQ ID NO: 31 - DNA, 28 bp, Salmonella sp.
  source 1..28: mol_type = genomic DNA
tttcctaacgtgttgtattgcatgactc

SEQ ID NO: 32 - DNA, 28 bp, Salmonella sp.
  source 1..28: mol_type = genomic DNA
tttacgcttttaacatgcggctagccgg

SEQ ID NO: 33 - DNA, 28 bp, Salmonella sp.
  source 1..28: mol_type = genomic DNA
tttcgcgttgcaggcaggcggcaagtta

SEQ ID NO: 34 - DNA, 28 bp, Salmonella sp.
  source 1..28: mol_type = genomic DNA
tttgattctttatttgtgataagggttt

SEQ ID NO: 35 - DNA, 28 bp, Salmonella sp.
  source 1..28: mol_type = genomic DNA
tttccagcagcagcgggaagttcaccgc

SEQ ID NO: 36 - DNA, 28 bp, Salmonella sp.
  source 1..28: mol_type = genomic DNA
tttatgtcgctggaattgggaggccggg

SEQ ID NO: 37 - DNA, 28 bp, Salmonella sp.
  source 1..28: mol_type = genomic DNA
tttgtgaatggtcgcccggactgggata

SEQ ID NO: 38 - DNA, 28 bp, Salmonella sp.
  source 1..28: mol_type = genomic DNA
tttctctttcagcgcctgcgctaaacgg

SEQ ID NO: 39 - DNA, 28 bp, Salmonella sp.
  source 1..28: mol_type = genomic DNA
tttaccttatattgaagttaagaaatag

SEQ ID NO: 40 - DNA, 28 bp, Salmonella sp.
  source 1..28: mol_type = genomic DNA
tttgcgcgcaaacggcatccgttaaaaa

SEQ ID NO: 41 - DNA, 28 bp, Salmonella sp.
  source 1..28: mol_type = genomic DNA
tttaatccctatggcaaaacgagagaga

SEQ ID NO: 42 - DNA, 28 bp, Salmonella sp.
  source 1..28: mol_type = genomic DNA
tttgttgcgtctgccgctgttttataac

SEQ ID NO: 43 - DNA, 28 bp, Salmonella sp.
  source 1..28: mol_type = genomic DNA
tttcgccaatgatttcccgctttacgac

SEQ ID NO: 44 - DNA, 28 bp, Salmonella sp.
  source 1..28: mol_type = genomic DNA
tttcgcgatcaagacgtttgagtaaaaa

SEQ ID NO: 45 - DNA, 28 bp, Salmonella sp.
  source 1..28: mol_type = genomic DNA
tttcatcataaggaacttcatagaaagc

SEQ ID NO: 46 - DNA, 28 bp, Salmonella sp.
  source 1..28: mol_type = genomic DNA
tttctcaaagttacgacttgcaaatatt

SEQ ID NO: 47 - DNA, 24 bp, Salmonella sp.
  source 1..24: mol_type = genomic DNA
attattgctcaattcgcaaaaatc

SEQ ID NO: 48 - DNA, 24 bp, Salmonella sp.
  source 1..24: mol_type = genomic DNA
agcactgattcagagttgagtacg

SEQ ID NO: 49 - DNA, 24 bp, Salmonella sp.
  source 1..24: mol_type = genomic DNA
atatgcggcggatgccactcctgg

SEQ ID NO: 50 - DNA, 24 bp, Salmonella sp.
  source 1..24: mol_type = genomic DNA
cgagcgcaggcaacgccgctgatg

SEQ ID NO: 51 - DNA, 24 bp, Salmonella sp.
  source 1..24: mol_type = genomic DNA
gaaggtttttgataaagcaatcct

SEQ ID NO: 52 - DNA, 24 bp, Salmonella sp.
  source 1..24: mol_type = genomic DNA
cgttctgatgtaatctgaatcagt

SEQ ID NO: 53 - DNA, 24 bp, Salmonella sp.
  source 1..24: mol_type = genomic DNA
cgagcgcaggcaacgccgctgatg

SEQ ID NO: 54 - DNA, 24 bp, Salmonella sp.
  source 1..24: mol_type = genomic DNA
accagcgagccgctgccggaaagg

SEQ ID NO: 55 - DNA, 24 bp, Salmonella sp.
  source 1..24: mol_type = genomic DNA
agcactgattcagattacatcaga

SEQ ID NO: 56 - DNA, 24 bp, Salmonella sp.
  source 1..24: mol_type = genomic DNA
gaaggtttttgataaagcaatcct

SEQ ID NO: 57 - DNA, 24 bp, Salmonella sp.
  source 1..24: mol_type = genomic DNA
accagcgagccgctgccggaaagg

SEQ ID NO: 58 - DNA, 24 bp, Salmonella sp.
  source 1..24: mol_type = genomic DNA
atatgcggcggatgccactcctgg

SEQ ID NO: 59 - DNA, 32 bp, synthetic construct
  source 1..32: mol_type = other DNA
tgatacgcagaccagaagacagacgctcgata

SEQ ID NO: 60 - DNA, 32 bp, synthetic construct
  source 1..32: mol_type = other DNA
tcaattgttgacacccgaatatacccaatagt

SEQ ID NO: 61 - DNA, 32 bp, synthetic construct
  source 1..32: mol_type = other DNA
tatcgccgctgcccagcgaatccgcatggtca

SEQ ID NO: 62 - DNA, 32 bp, synthetic construct
  source 1..32: mol_type = other DNA
tgagctgatcgtgaccggcgataacaactaca

SEQ ID NO: 63 - DNA, 32 bp, synthetic construct; identical to SEQ ID NO: 1
  source 1..32: mol_type = other DNA

SEQ ID NO: 64 - DNA, 32 bp, synthetic construct; identical to SEQ ID NO: 2
  source 1..32: mol_type = other DNA